(19) United States
(12) Patent Application Publication
(10) Pub. No.: US 2026/0041456 A1
(43) Pub. Date: Feb. 12, 2026

(54) Trackable Surgical Dilator System

(71) Applicant: See All AI Inc., Nashua, NH (US)

(72) Inventors: Michael Craig Barrus, Redondo Beach, CA (US); Ryan Gregory Woods, Broomfield, CO (US); Lisa Ung Cavaliere, Ramsey, NJ (US)

(73) Assignee: See All AI Inc., Nashua, NH (US)

(21) Appl. No.: 19/279,905

(22) Filed: Jul. 24, 2025

Related U.S. Application Data
(60) Provisional application No. 63/675,178, filed on Jul. 24, 2024.

(51) Int. Cl. A61B 17/34; A61B 17/00; A61B 34/20; A61B 90/00

(52) U.S. Cl. CPC A61B 17/3472; A61B 17/3421; A61B 34/20; A61B 90/08; A61B 2017/00477; A61B 2017/3456; A61B 2034/2055; A61B 2090/0807

(57) ABSTRACT

A trackable dilator system that provides visual confirmation as its component configurations utilized a tubular stylus having an end configured for securing to one of a plurality of topper elements, each being visually discernible from others of the plurality of topper elements because of one of a different color, shape, reflectance, luminance, or material composition properties of the topper element. Also disclosed is a dilator system having a proximal exterior with a maximum outer diameter relative to an interior lumen. A plurality of subregions of the exterior region are characterized by a dilator wall thickness less than the maximum outer diameter to facilitate visual recognition of the axis and depth of the dilator body when in situ.

### FIELD OF INVENTION

The disclosure relates to orthopedic surgery and specifically to a trackable tool to a hole into the pedicle of a spinal vertebra.

### BACKGROUND

The current state of the art for creating a hole in the pedicle of a vertebral body is to use a bone marrow biopsy needle assembly or a ‘Jamshidi’, as illustrated in the prior art system of FIG. 1. The needle has a handle and is cannulated to receive a stylet that has a trocar point. The stylet attaches to the needle via a Luer-lock. The assembly with trocar point is impacted into the bone. Unfortunately, such needle assemblies have limitations of size and the handle can be obtrusive in certain surgical procedures. Often the position and angle of orientation of the biopsy needle cannot be clearly determined from medical images. In addition, it is not always possible to determine form visual observation, by camera, or otherwise, what stylet or wire is currently used with the needle assembly.

Accordingly, need exists for a more versatile and trackable dilator for certain surgical procedures.

A further need exists for a more versatile and trackable dilator for creating a hole in the pedicle of a vertebral body.

A still further need exists for a more versatile and trackable dilator whose position and angle of orientation can be more readily determined from medical images.

A still further need exists for a trackable dilator system from provides visual confirmation as to the configuration the stylus and stylet combination being utilized.

### SUMMARY OF THE INVENTION

According to one aspect of the disclosure, a dilator system comprises kit for use with a surgical navigation system, the kit comprising: a dilator in the form of a tubular stylus having an interior lumen extending between a first tapered end thereof and a second end thereof configured for securing to a topper element; a plurality of topper elements, each topper element securable to the second end of the tubular stylus and being visually discernable from others of the plurality of topper elements because of a different color, shape, reflectance, luminance, material composition, or combination thereof, of exterior surfaces of the topper element. In embodiments, the plurality of topper elements have a similar shape but visually discernable from others of the plurality of topper elements because of any of a different color property, different surface reflectance property, different surface texture, different material composition, different surface luminance property. In embodiments, one of the plurality of topper elements is secured to a trocar tip stylet or blunt tip stylet receivable within the lumen of the tubular stylus. In embodiments, one of the plurality of topper elements has a lumen extending therethrough which is in fluid communication with the lumen of the tubular stylus when the topper element is secured to the second end of the tubular stylus, thereby forming a combined lumen therewith from the first end of the tubular stylus to exteriorly of the one topper element secured to the second end of the tubular stylus. In embodiments, the tubular stylus has measuring indicia on an exterior surface thereof distance. In embodiments, the tubular stylus has exterior features proximal of the first end which define a maximum outer diameter and facilitate visual recognition of the axis and depth of the dilator body when in situ. In embodiments, the tubular stylus has an exterior region proximal of the first end having a maximum outer diameter relative to the interior lumen, wherein a plurality of subregions of the exterior region are characterized by a wall thickness less than the maximum outer diameter.

According the another aspect of the disclosure, disclosed is a trackable dilator tool to a hole into the pedicle of a spinal vertebra. The disclosed tool is dilator system characterized by its distinct external geometry that enables its precise position and depth to be determined under visible light.

According to one aspect of the disclosure, a dilator system comprises a cylindrical dilator body having a lumen extending therethrough, the dilator body having a tapered distal end and a more proximal outer diameter region characterized by a repeating pattern of progressively increasing diameter and diameter decreases. In one embodiment, the dilator is used in combination with a stylet having an outer diameter sized to be received within the lumen of the dilator. In one embodiment, a proximal end of the dilator and a proximal end of the stylet have complementary interlocking features enabling the dilator and stylet to be securable together. In one embodiment, the dilator body has a generally circular cross-sectional profile and the outer diameter has a feature that changes pitch after every revolution of the circular cross-sectional profile.

According to another aspect of the disclosure, a surgical dilator comprises: a dilator wall defining an elongate cylindrical dilator body having an interior lumen, a tapered distal end, and an exterior region proximal of the distal end having a maximum outer diameter relative to the interior lumen, wherein a plurality of subregions of the exterior region are characterized by a dilator wall thickness less than the maximum outer diameter.

According to another aspect of the disclosure, a surgical dilator comprises: an elongate cylindrical dilator body having an interior lumen extending along an axis thereof and a tapered distal end, wherein the dilator body has exterior features proximal of the distal end which define a maximum outer diameter and facilitate visual recognition of the axis and depth of the dilator body when in situ.

### DESCRIPTION OF THE DRAWINGS

The various features and advantages of the disclosure may be more readily understood with reference to the following detailed description taken in conjunction with the accompanying drawings, wherein like references designate like structural elements.

FIG. 1 illustrates conceptually a prior art bone marrow biopsy needle assembly

FIG. 2A illustrates conceptually a perspective view of a dilator system including a dilator and stylet in accordance with the disclosure.

FIG. 2B illustrates conceptually a perspective view of the dilator system of FIG. 2A with the stylet inserted in the dilator in accordance with the disclosure.

FIG. 3 is a plan view of the dilator system of FIG. 2A with the stylet inserted in the dilator in accordance with the disclosure.

FIG. 4 is a plan cut away view of the dilator system of FIG. 3 as taken along line A-A in accordance with the disclosure.

FIG. 5 is an end cut away view of the dilator system of FIG. 3 as taken along line C-C in accordance with the disclosure.

FIG. 6 is an end cut away view of the dilator system of FIG. 3 as taken along line C′-C′ in accordance with the disclosure.

FIG. 7 is an end cut away view of the dilator system of FIG. 3 as taken along line C″-C″ in accordance with the disclosure.

FIG. 8 illustrates conceptually a perspective view of a dilator system of FIG. 2A with the stylet partially withdrawn from the dilator in accordance with the disclosure.

FIG. 9 is an enlarged view of section B of FIG. 8 illustrating the locking features of the stylet and dilator in accordance with the disclosure.

FIG. 10 illustrates conceptually a perspective view of a dilator system including a dilator and stylet in accordance with the disclosure.

FIG. 11 is a plan cut away view of the dilator system of FIG. 10 as taken along line A-A in accordance with the disclosure.

FIG. 12 is an end cut away view of the dilator system of FIG. 11 as taken along line C-C in accordance with the disclosure.

FIG. 13 is an end cut away view of the dilator system of FIG. 11 as taken along line C′-C′ in accordance with the disclosure.

FIG. 14 is an end cut away view of the dilator system of FIG. 11 as taken along line C″-C″ in accordance with the disclosure.

FIG. 15 illustrates conceptually a perspective view of a dilator system with the stylet partially withdrawn from the dilator in accordance with the disclosure.

FIG. 16 is an enlarged view of section B of FIG. 15 illustrating the locking features of the stylet and dilator.

FIG. 17 illustrates conceptually a perspective view of a dilator system including multiple implements and visually discernible and interchangeable topper elements in accordance with the disclosure.

FIG. 18 illustrates conceptually a perspective view of a dilator system including a topper element capable of receiving a guidewire in accordance with the disclosure.

FIG. 19 illustrates conceptually a perspective view of a topper element capable in accordance with the disclosure.

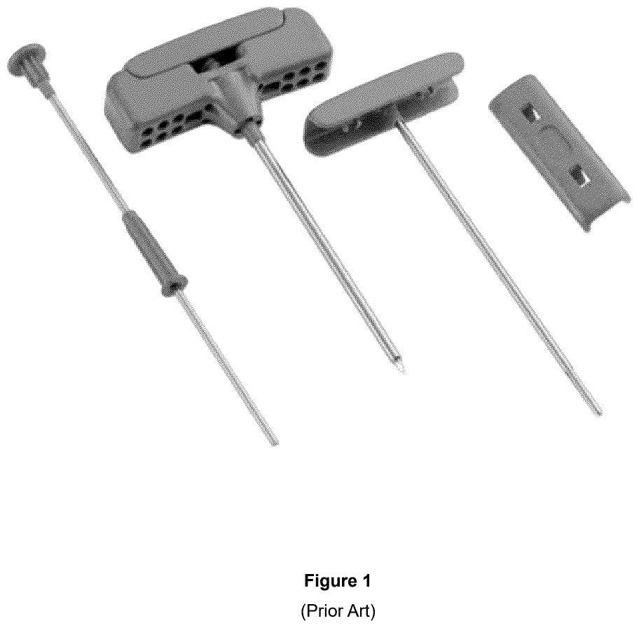

### DETAILED DESCRIPTION

The present disclosure will be more completely understood through the following description, which should be read in conjunction with the drawings. In this description, like numbers refer to similar elements within various embodiments of the present disclosure. The skilled artisan will readily appreciate that the methods, apparatus and systems described herein are merely exemplary and that variations can be made without departing from the spirit and scope of the disclosure.

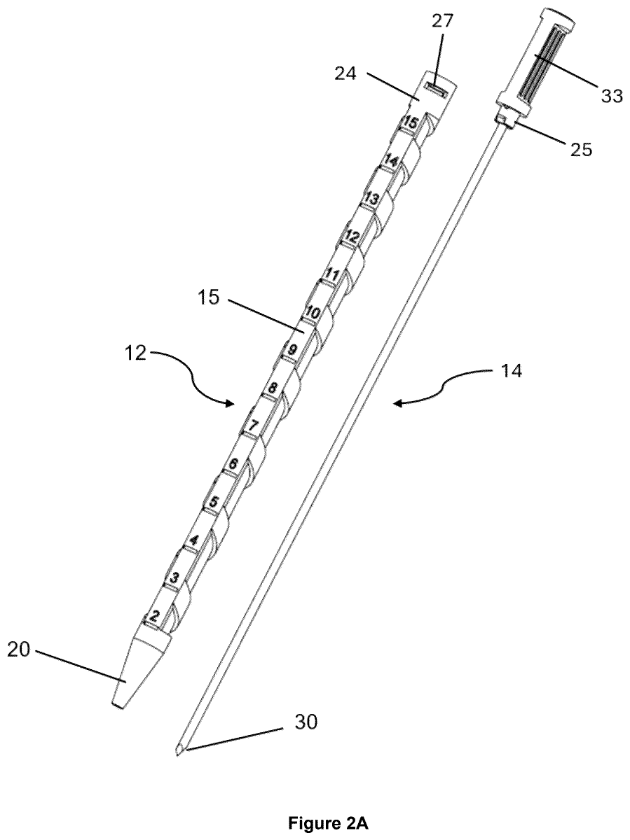

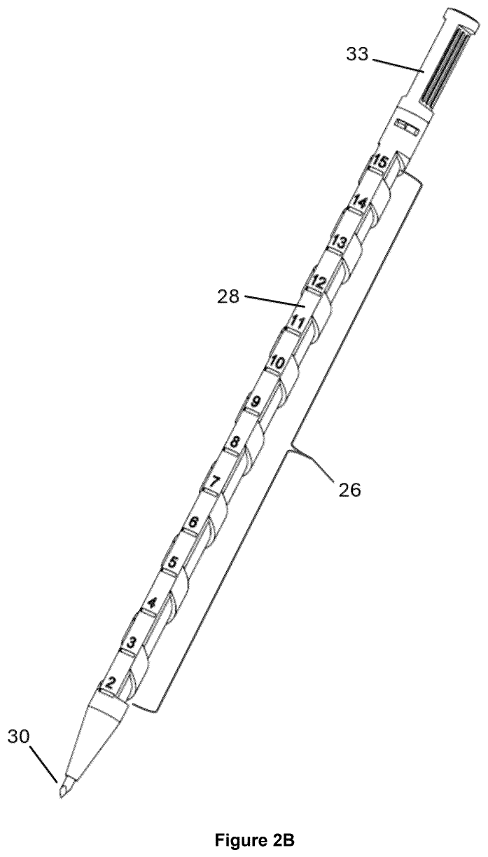

FIG. 2A illustrates a dilator system 10 comprising a dilator 12 and a stylet 14. FIG. 2A illustrates conceptually a perspective view of a dilator system 10 including a dilator 12 and stylet 14. The dilator comprises an elongate, cylindrical dilator body 15 defined by a dilator wall 17. The dilator wall defines an interior lumen 16 extending through the dilator body 15 and is sized to accommodate a stylet 14 insertable therein. The dilator wall 17 has a tapered exterior diameter 18 at a first or distal end 20 of the dilator body 15. The dilator wall 17 further defines a dilator maximum outer diameter 22 in a region more proximal than the first end 20, as explained in greater detail herein.

In embodiments, the dilator body 15 may comprise any number of substantially rigid biocompatible plastics or resins having adequate columnar strength to axially transmit force and retain the stylet 14 securely within lumen 16. In embodiments, the dilator 12 may be made of a single-use polymer. In embodiments, the dilator 12 may have an approximately 8 mm maximum outer diameter, i.e. 24 French, and be approximately 200 mm long. In embodiments, the interior lumen 16 may of dilator body 15 be sized and shaped to accept the outer diameters of any number of commercially available stylet implementations.

FIG. 2B illustrates conceptually a perspective view of the dilator system 10 of FIG. 2A with the stylet 14 inserted in the dilator 12. The stylet 14 is insertable into the interior lumen 16 at a second or proximal end 24 of the dilator body 15 and is removably securable to the dilator body 15 using a co-acting interacting locking mechanism 25 disposed at the proximal end 24 of the dilator body 15 and the stylet 14, as illustrated.

In embodiments, stylets 14 having any variety of distal tip configurations and may be used with the disclosed dilator 12 as long as they can be accommodated within the interior lumen 16 extending through the dilator body 15. The stylet 14 may have a traditional trocar design with a pointed distal tip 30 for impacting bone and may be made from any number of biocompatible metals, such as surgical stainless steel, or sufficiently rigid polymers with adequate columnar strength. The stylet 14 may also have a traditional a blunt distal tip 32, as illustrated in Figure and may be similarly made from any number of biocompatible metals, such as surgical stainless steel, or sufficiently rigid polymers with adequate columnar strength.

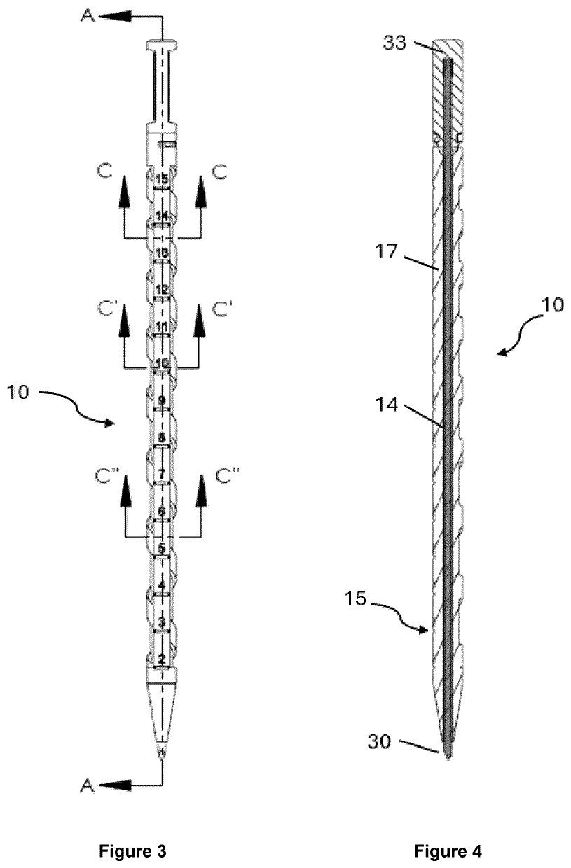

According to one aspect of the disclosure, as can be seen in FIGS. 3-7 and 10-14, the dilator 12 may have maximum outer diameter 22 but with a non-uniform or varying thickness of dilator wall 17 defining the dilator body 15.

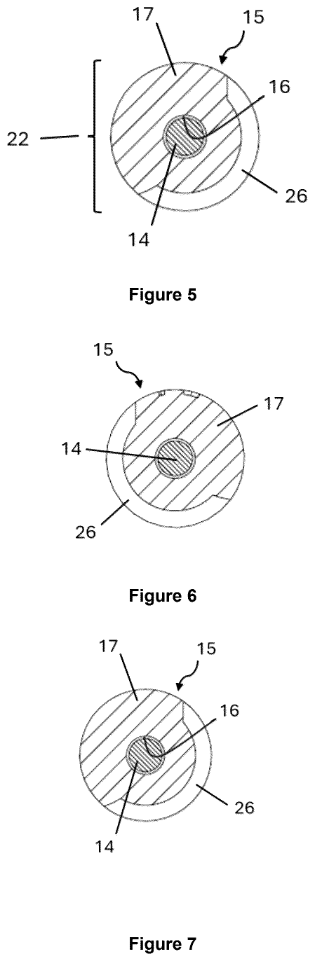

FIGS. 3 and 4 illustrate another dilator system 10 comprising a dilator 12 and a stylet 14 with the dilator 12 may be similar in construction and function to that described with reference to FIGS. 2A and B, except that the exterior of the dilator body 15 has the exterior features as shown in FIGS. 3-7. In embodiments, as illustrated in FIGS. 3-7, the outer diameter 22 defining the exterior of the dilator body 15 may be implemented with an exterior helical surface feature 26, such as a machine thread with a progressive helix, e.g. the outer diameter changes pitch with revolution along the dilator body 15. FIG. 3 is a plan view of the dilator system 12 with the stylet 14 inserted in the dilator. FIG. 4 is a plan cut away view of the dilator system 12 of FIG. 3 as taken along line A-A. FIG. 5 is an end cut away view of the dilator system of FIG. 3 as taken along line C-C. FIG. 6 is an end cut away view of the dilator system of FIG. 3 as taken along line C′-C. FIG. 7 is an end cut away view of the dilator system of FIG. 3 as taken along line C″-C″. As such, the dilator 12 has a visually discernable effective orientation along an axis of the lumen 16, enabling cameras of a surgical navigation system to observe the direction and positional orientation of the dilator 12, even when full view of the dilator body 15 is partially obstructed and to facilitate visual recognition of the axis and depth of the dilator body when in situ. In embodiments, the dilator 12 may have a maximum outer diameter 22 which allows for use with larger diameter dilator systems.

One or more exterior surfaces of the dilator body 15 of FIGS. 3 and 4, may have a series of reference indicia 28 such as tick marks, numbers, indentations, or any combination thereof, to enable a practitioner to determine distances or axial portions of the dilator length relative to its surrounding context, as illustrated.

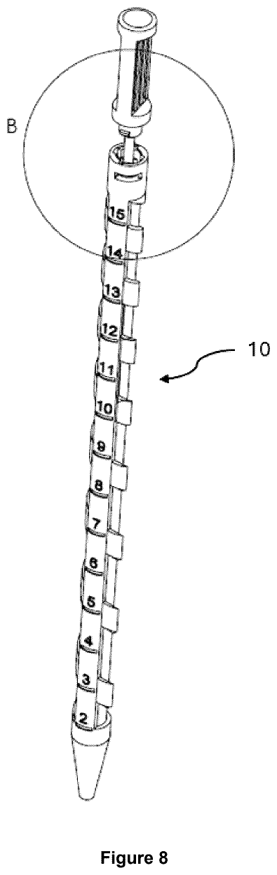

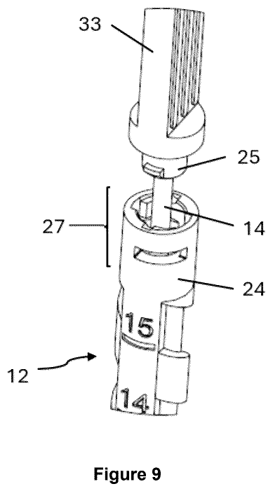

The proximal end of the stylet 14 includes a handle 33 securely fixed to the stylet body, as illustrated in FIGS. 8 and 9. In embodiments, the stylet handle 33 may have a generally cylindrical shape of varying stepped diameters, one end of which fits securely into the proximal end 24 of the dilator body 15. One or more projections on the handle 33 of stylet 14 is received within a complementary mating track on the inner diameter at the proximal end 24 of the dilator body 15. A turn of the stylet handle relative to the dilator body 15 enables removably locking or unlocking of the stylet 14 and dilator 12. FIG. 8 illustrates conceptually a perspective view of a dilator system of FIG. 2A with the stylet 14 partially withdrawn from the dilator 12. FIG. 9 is an enlarged view of section B of FIG. 8 illustrating the locking features 25 and 27 of the stylet 14 and dilator 12, respectively.

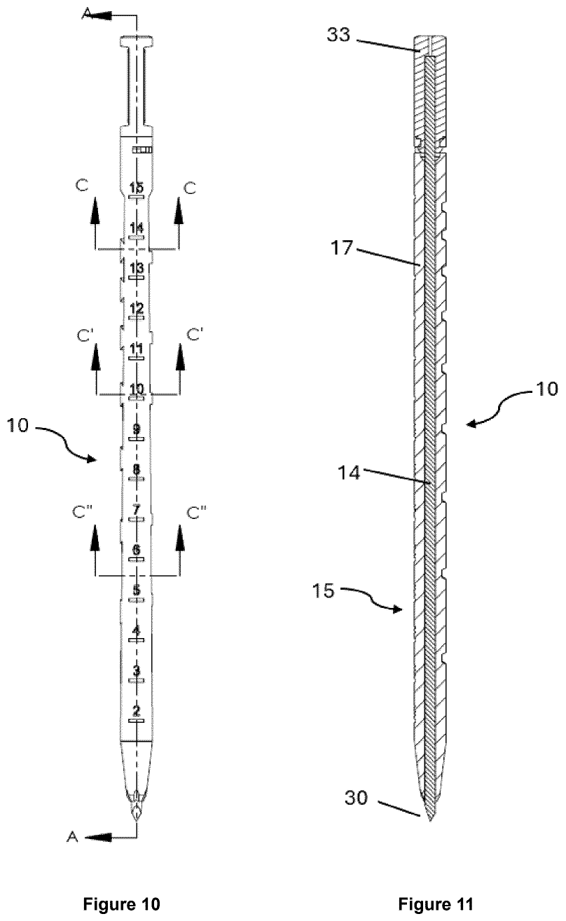

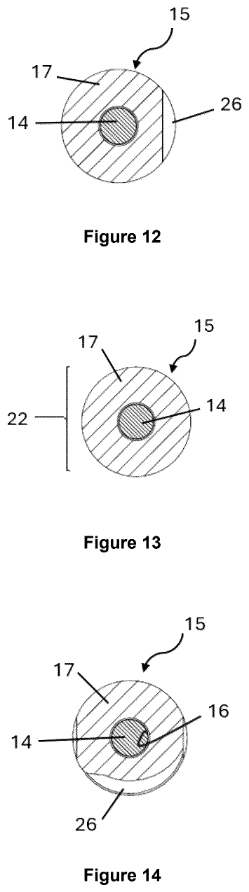

FIGS. 10 and 11 illustrate another dilator system 10 comprising a dilator 12 and a stylet 14 with the dilator 12 may be similar in construction and function to that described with reference to FIGS. 2A and B, except that the exterior of the dilator body 15 has the exterior features as shown in FIGS. 10-15. FIG. 10 illustrates conceptually a perspective view of a dilator system 10 including a dilator 12 and stylet 14. FIG. 11 is a plan cut away view of the dilator system 10 of FIG. 10 as taken along line A-A. As illustrated, the distal end 20 of the dilator wall 15 is tapered and includes radially and proximally extending indentations or ridges 19 to facilitate coring into the patient anatomic features. In addition, as can be seen in FIGS. 10-14, the dilator wall 17 further defines a maximum outer diameter 22 of the dilator body 15 in a region more proximal than the first end 20, as well as a number of areas with a wall diameter less than the maximum outer diameter 22, as illustrated. FIG. 12 is an end cut away view of the dilator system 10 of FIG. 11 as taken along line C-C. FIG. 13 is an end cut away view of the dilator system 10 of FIG. 11 as taken along line C′-C′. FIG. 14 is an end cut away view of the dilator system 10 of FIG. 11 as taken along line C″-C″. As such, the dilator 12 has a visually discernable effective orientation along an axis of the lumen 16, enabling cameras of a surgical navigation system, to observe the direction and positional orientation of the dilator 12, even when full view of the dilator body 15 is partially obstructed and to facilitate visual recognition of the axis and depth of the dilator body when in situ.

Similar to the embodiment of FIGS. 2A-B and 3, the dilator body 15 of FIGS. 10-14, may have an on the exterior surface thereof a series of reference indicia such as tick marks, numbers, indentations, or any combination thereof, to enable a practitioner to determine axial portions of the dilator length relative to its surrounding context.

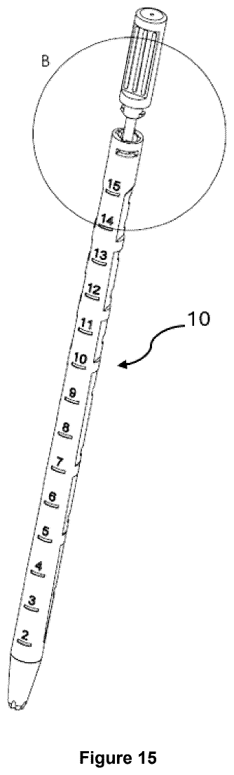

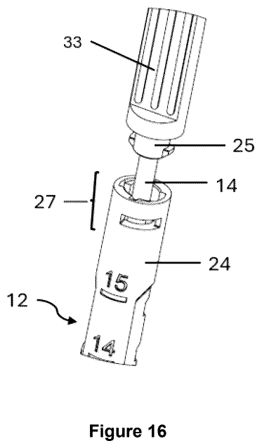

The proximal end of the stylet 14 includes a handle securely fixed to the stylet body, similar to that previously described herein, and is removably securable to dilator 12 in a manner a s previously described, as illustrated in FIGS. 15-16. FIG. 15 illustrates conceptually a perspective view of a dilator system 10 with the stylet 14 partially withdrawn from the dilator 12. FIG. 16 is an enlarged view of section B of FIG. 15 illustrating the locking features 25 and 27 of the stylet 14 and dilator 12, respectively.

In use, the pieces of the dilator system 10 are removed from a sterile blister pack kit and the stylet 14 inserted into the dilator 10 while aligning the locking feature 25 on the stylet handle to the complementary locking feature 27 at second end 24 of dilator 12. Thereafter, the stylet handle may be turned, e.g. one quarter revolution clockwise, into the dilator 12 to lock the stylet 14 into position. The instrument is navigable, and the system will guide it into position. The distal tapered end 18 of the dilator 12 allows dissection of tissue as the dilator is pushed into position. Impacting the distal end 20 of the dilator 12 will seat the trocar tip 30 of the stylet 14 into bone and create a hole. Once positioned, stylet handle may be counter turned to unlock and disengage the stylet 14 from the dilator 12. While maintaining the position of dilator 12, a guidewire 40 may then be inserted down into lumen 16. The cylindrical design dilator 12 and the alternating maximum outer diameter, as described herein, allows for a sequentially larger lumened dilator to be used in conjunction with system 10 by sliding the larger cannulated dilator over the proximal end 24 and remove the dilator 12 once positioned.

Alternatively, the dilator 12 may be used without the stylet 14 inserted. The dilator 15 can be used as a pointer to navigate patient anatomy. Once the anatomic feature is located a guidewire 40 may be inserted into lumen 16 of the dilator 12 and driven into the patient anatomy, as described in further detail herein.

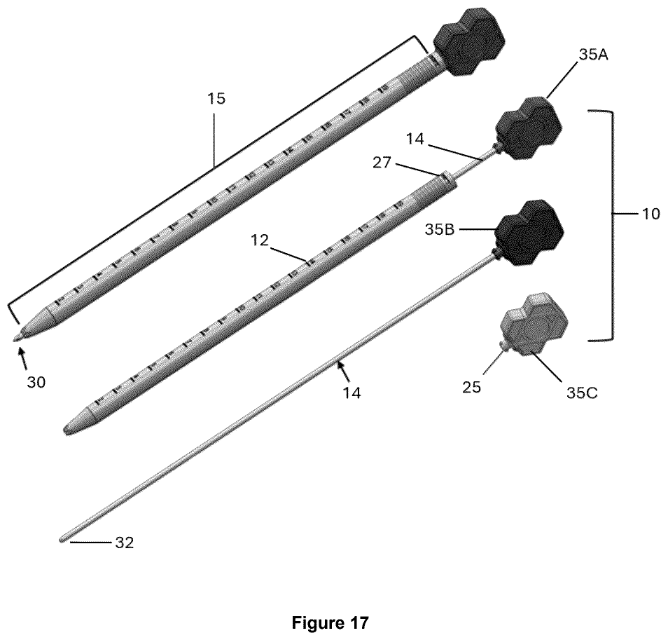

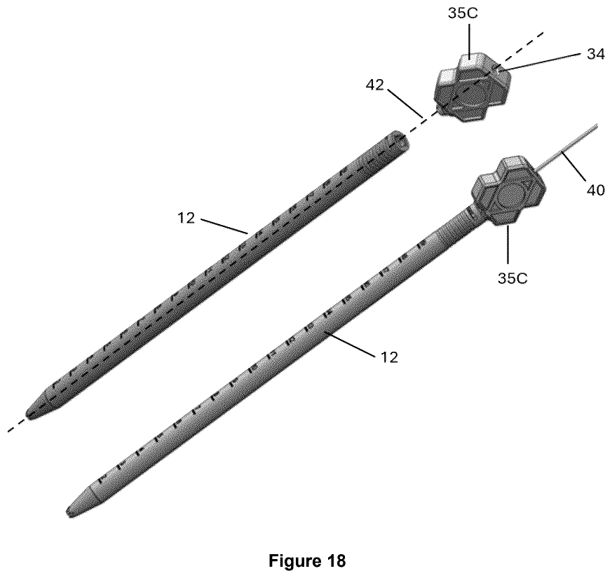

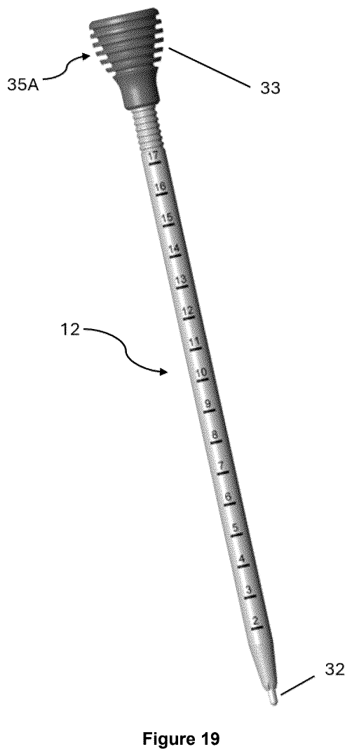

According to another aspect of the disclosure, a dilator system 10 comprises kit for use with a surgical navigation system, as illustrated with reference to FIGS. 17 and 18. FIG. 17 illustrates conceptually a perspective view of a dilator system including a dilator and multiple implements having visually discernible and interchangeable topper elements. FIG. 19 illustrates conceptually a perspective view of a dilator system including a topper element capable of receiving a guidewire.

In embodiments, the kit comprises a dilator 12 and a plurality topper elements 35 securable to the end 24 of dilator 12. In embodiments, the dilator 12 may be similar in construction and function to that described with reference to FIG. 2A and B, except that the exterior of the dilator body 15 may or may not have the variations in the thickness of the dilator wall 17 as described herein.

In embodiments, each topper element 35A-C is securable to the second end 24 of dilator 12 in a manner as previously described herein. In embodiments, the exterior surfaces of topper element 35A-C are visually discernable from others of the plurality of topper elements because of a difference in appearance caused by varying any of a different color, shape, reflectance, luminance, material composition, or combination thereof, of such exterior surfaces among the topper element. In embodiments, each topper element 35A-C has a body member with a shape defined by exterior surfaces thereof. In embodiments, one surface of each topper element body member has an interlock mechanism 25, which may be similar to that previously described herein, for interacting with a complementary interlocking mechanism 27 on the second end 24 of dilator 12. It will be obvious to those reasonably skilled in the relevant arts that other interlocking mechanisms or securing schemes may be equivalently used to removably secure a topper element to dilator 12. In embodiments, may have all or only a portion of their respective exterior surfaces visually differentiated to appear distinct. In embodiments, the plurality of topper elements 35A-C, as illustrated in FIG. 17, have a similar shape but are visually discernable from each other because each topper element is has a different color on exterior surfaces thereof. Topper elements 35A, having a green or first color exterior surface, is secured to a stylet having a pointed trocar tip 30. Topper elements 35B, having a black or second color exterior surface, is secured to a stylet having a blunt tip 32.

Topper elements 35C, having a grey or third color exterior surface, has an internal lumen 34 preferably of similar diameter to lumen 16 of dilator 12, extending through the body member thereof, for receiving a guidewire 40 or other surgical implement therein. When properly secured, lumen 34 of topper element 35C is in fluid communication with lumen 16 of dilator 12, thereby forming a combined lumen extending from the first end 20 of dilator 12 to exteriorly of topper element 35C along axis 42 in FIG. 18.

In embodiments, the topper elements 35A-C may have all or only a portion of their respective exterior surfaces visually differentiated from each other to appear distinct. In embodiments, topper elements 35A-C may have a similar shape but different surface reflectance properties, or different surface textures, or different surface luminance properties, or different material composition which result in a combination of any of the foregoing properties to visually differentiate the topper elements from each other.

At various places in the present specification, values are disclosed in groups or in ranges. It is specifically intended that the description includes each and every individual sub-combination of the members of such groups and ranges and any combination of the various endpoints of such groups or ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

For purposes of clarity and a concise description, features are described herein as part of the same or separate embodiments, however, it will be appreciated that scope of the concepts may include embodiments having combinations of all or some of the features described herein.

It will be apparent to those recently skilled in the art that modifications to the apparatus and process disclosed here in may occur, including substitution of various component values or nodes of connection, without parting from the true spirit and scope of the disclosure as defined by the claims set forth herein.

What is claimed is:

1. A kit of surgical or diagnostic implements for use with a surgical navigation system having at least one camera, the kit comprising:
a tubular stylus having a lumen extending between a first tapered end thereof and a second end configured for securing to a topper element;
a plurality of topper elements, each topper element securable to the second end of the tubular stylus and being visually discernable from others of the plurality of topper elements because of one of a different color, shape, reflectance, luminance, or material composition properties of the topper element.

2. The kit of claim 1 wherein the plurality of topper elements have a similar shape but visually discernable from others of the plurality of topper elements because of a different color property.

3. The kit of claim 1 wherein the plurality of topper elements have a similar shape but are visually discernable from others of the plurality of topper elements because of a different surface reflectance property.

4. The kit of claim 1 wherein the plurality of topper elements have a similar shape but visually discernable from others of the plurality of topper elements because of a different surface texture.

5. The kit of claim 1 wherein the plurality of topper elements have a similar shape but visually discernable from others of the plurality of topper elements because of a different material composition.

6. The kit of claim 1 wherein the plurality of topper elements have a similar shape but visually discernable from others of the plurality of topper elements because of a different surface luminance property.

7. The kit of claim 1 wherein one of the plurality of topper elements is secured to a trocar tip stylet receivable within the lumen of the tubular stylus.

8. The kit of claim 1 wherein one of the plurality of topper elements is secured to a blunt tip stylet receivable within the lumen of the tubular stylus.

9. The kit of claim 1 wherein one of the plurality of topper elements has an lumen extending therethrough which is in fluid communication with the lumen of the tubular stylus when the topper element is secured to the second end of the tubular stylus.

10. The kit of claim 1 wherein the one of the plurality of topper elements when secured to the tubular stylus forms a continuous lumen therewith from the first end of the tubular stylus to exteriorly of the one topper element.

11. The kit of claim 1 wherein the tubular stylus has distance measuring indicia on an exterior surface thereof.

12. A surgical dilator comprising:
a dilator wall defining an elongate cylindrical dilator body having an interior lumen, a tapered distal end, and an exterior region proximal of the distal end having a maximum outer diameter relative to the interior lumen,
wherein a plurality of subregions of the exterior region are characterized by a dilator wall thickness less than the maximum outer diameter.

13. The dilator of claim 12 in combination with a stylet having an outer diameter sized to be received within the lumen of the dilator.

14. The dilator of claim 12 wherein the dilator and stylet are securable together.

15. The dilator of claim 14 wherein a proximal end of the dilator and a proximal end of the stylet have complementary interlocking features.

16. The dilator claim 15 wherein the dilator body has a generally circular cross-sectional profile and the outer diameter has a feature that changes pitch after every revolution of the circular cross-sectional profile.

17. A surgical dilator comprising:
an elongate cylindrical dilator body having an interior lumen extending along an axis thereof and a tapered distal end,
wherein the dilator body has exterior features proximal of the distal end which define a maximum outer diameter and facilitate visual recognition of the axis and depth of the dilator body when in situ.

18. The dilator of claim 17 in combination a plurality of topper elements, each topper element securable to the second end of the dilator body and being visually discernable from others of the plurality of topper elements because of one of a different color, shape, reflectance, luminance, or material composition properties of the topper element.

19. The dilator of claim 18 wherein one of the plurality of topper elements has an lumen extending therethrough which is in fluid communication with the lumen of the tubular stylus when the topper element is secured to the second end of the dilator.

20. The dilator of claim 19 wherein the one of the plurality of topper elements when secured to the tubular stylus forms a continuous lumen therewith from the first end of the dilator to exteriorly of the one topper element.
